(19) United States
(12) Patent Application Publication
(10) Pub. No.: US 2025/0180480 A1
(43) Pub. Date: Jun. 5, 2025

(54) DETERMINING PARTITION COEFFICIENTS OF TRACER ANALYTES

(71) Applicant: Saudi Arabian Oil Company, Dhahran (SA)

(72) Inventors: Sehoon Chang, Boston, MA (US); Gawain Thomas, Shirley, MA (US); Wei Wang, Quincy, MA (US); Hooisweng Ow, Woburn, MA (US)

(21) Appl. No.: 19/050,214

(22) Filed: Feb. 11, 2025

Related U.S. Application Data
(62) Division of application No. 17/643,983, filed on Dec. 13, 2021, now Pat. No. 12,253,467.

(51) Int. Cl. G01N 21/64; G01N 1/38; G01N 33/18

(52) U.S. Cl. CPC G01N 21/64; G01N 1/38; G01N 33/18

(57) ABSTRACT

Optical properties of a tracer in water are measured at varying concentrations. A reference curve is built based on the measured optical properties at varying concentrations. An emulsion is mixed with the tracer. The emulsion is demulsified into an oil component and an aqueous component. Optical properties of one of the components are measured. A partition coefficient is determined based on the measured optical properties of a demulsified component and the reference curve.

### CROSS-REFERENCE TO RELATED APPLICATIONS

This application is a divisional of and claims the benefit of priority to U.S. patent application Ser. No. 17/643,983, filed Dec. 13, 2021, the contents of which are incorporated by reference herein.

### TECHNICAL FIELD

This disclosure relates to determining partition coefficients.

### BACKGROUND

The Environmental Protection Agency (EPA) uses partition coefficients to predict the environmental fate, aquatic toxicity, and bioaccumulation of chemicals and pollutants. The partition coefficient of tracers for oil reservoirs is defined by the ratio of tracer concentrations in the oil and water phases at equilibrium.

The partition coefficient is defined as the ratio of concentrations of a compound in a mixture of two immiscible solvents at equilibrium. Several methods for determining the partition coefficient are commonly used, for example, the static or “shake-flask” method, and the coreflooding method.

### SUMMARY

This disclosure describes technologies relating to determining partition coefficients of tracer analytes.

An example of the subject matter described within this disclosure is a method with the following features. Optical properties of a tracer in water are measured at varying concentrations. A reference curve is built based on the measured optical properties at varying concentrations. An emulsion is mixed with the tracer. The emulsion is demulsified into an oil component and an aqueous component. Optical properties of one of the components are measured. A partition coefficient is determined based on the measured optical properties of a demulsified component and the reference curve.

Aspects of the example method, which can be combined with the example method alone or with other aspects, can include the following. The measured component is the aqueous component.

Aspects of the example method, which can be combined with the example method alone or with other aspects, can include the following. Mixing includes static mixing.

Aspects of the example method, which can be combined with the example method alone or with other aspects, can include the following. Mixing the emulsion with the tracer includes receiving the emulsion and the trace by a microfluidic chip.

Aspects of the example method, which can be combined with the example method alone or with other aspects, can include the following. Demulsifying the emulsion includes flowing the mixed emulsion and tracer through a membrane separator.

Aspects of the example method, which can be combined with the example method alone or with other aspects, can include the following. The optical properties include ultraviolet visibility or fluorescence intensity.

Aspects of the example method, which can be combined with the example method alone or with other aspects, can include the following. The tracers include at least of one of the following: dipicolinic acid, chelidamic acid, 4-chloropyridine-2, 6-dicarboxylic acid, 1,5-naphthalenedisulfonate, 2-fluorobenzoic acid, or 4-Chlorobenzyl alcohol.

Aspects of the example method, which can be combined with the example method alone or with other aspects, can include the following. Measuring the optical properties includes exposing a sample to light at 214 nanometer wavelength.

Aspects of the example method, which can be combined with the example method alone or with other aspects, can include the following. Measuring the optical properties comprises measuring a visibility or fluorescent intensity of the sample responsive to exposing the sample to light.

An example of the subject matter described within this disclosure is a system with the following features. A microfluidic mixing chip, a separator, an optical detection system, and a controller are all included. The controller is configured to send a signal to a separation system. The signal is a command to begin a separation process. The controller is configured to receive a measurement signal from an optical detector. The measurement signal is indicative of a concentration of a tracer within a sample. The controller is configured to compare the measurement signal to a known dataset of tracer concentrations. The controller is configured to determine a partition coefficient based on the comparison of the measurement signal with the dataset.

Aspects of the example system, which can be combined with the example system alone or with other aspects, can include the following. The controller is further configured to activate a light source emitting a light at 214 nanometer wavelength or 254 nanometer wavelength.

Aspects of the example system, which can be combined with the example system alone or with other aspects, can include the following. The separator is a membrane separator.

Aspects of the example system, which can be combined with the example system alone or with other aspects, can include the following. The optical detection system includes a light sensor.

Aspects of the example system, which can be combined with the example system alone or with other aspects, can include the following. The optical detection system includes a controllable light source emitting light at 254 nanometer wavelength.

An example of the subject matter described within this disclosure is a method with the following features. Optical properties of an aqueous tracer in water are measured at varying concentrations. Based on the measured optical properties at varying concentrations, a reference curve is developed. An emulsion is mixed with the aqueous tracer by a microfluidic chip. The emulsion is demulsified by a membrane separator. Optical properties of a demulsified component are measured. A partition coefficient is determined based on the measured optical properties of the demulsified components and the developed reference curve.

Aspects of the example method, which can be combined with the example method alone or with other aspects, can include the following. Measuring the optical properties includes exposing a sample to light at 214 nanometer wavelength or 254 nanometer wavelength.

Aspects of the example method, which can be combined with the example method alone or with other aspects, can include the following. Measuring the optical properties include measuring a visibility or fluorescent intensity of the sample responsive to exposing the sample to light.

Aspects of the example method, which can be combined with the example method alone or with other aspects, can include the following. The optical properties include ultraviolet visibility or fluorescence intensity.

Aspects of the example method, which can be combined with the example method alone or with other aspects, can include the following. The tracers include at least of one of the following: dipicolinic acid, chelidamic acid, 4-chloropyridine-2, 6-dicarboxylic acid, 1,5-naphthalenedisulfonate, 2-fluorobenzoic acid, or 4-Chlorobenzyl alcohol.

Aspects of the example method, which can be combined with the example method alone or with other aspects, can include the following. The demulsified component is an aqueous component.

Particular implementations of the subject matter described in this disclosure can be implemented so as to realize one or more of the following advantages. The subject matter described herein allows for rapid determination of partition coefficients. The subject matter described herein can be used determine partition coefficients with much less solvent and analyte usage compared to the conventional methods.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a flowchart of a method that can be used with aspects of this disclosure.

FIG. 2 is an example reference curve that can be used with aspects of this disclosure.

FIG. 3 is a schematic diagram of an example analysis system that can be used with aspects of this disclosure.

FIG. 4 is a schematic diagram of an example controller that can be used with aspects of this disclosure.

Like reference numbers and designations in the various drawings indicate like elements.

### DETAILED DESCRIPTION

To save time and cost, microfluidic methods allow for a fast, portable, solventless, and sustainable device for determining partition coefficients. Molecular transport of the analyte between two immiscible phases is often controlled by diffusion. By decreasing the characteristic length of diffusion, time required to complete the experiment is also reduced. The time scale is imposed by the short diffusion length of the analyte across two phases and large specific interfacial area.

This disclosure describes a system and method for quickly determining partition coefficients of desired analytes with significantly reduced time compared to traditional methods, such as the shaker method. The system and methods include mixing an emulsion and a tracer upon a microfluidic chip. The emulsification is then demulsified and the concentration of the tracer is measured in either the water phase or the oil phase (depending on which analyte is used). An optical property of the tracer is measured with an optical detector. The measurements detected by the optical detector are then compared to known optical measurements to determine a partition coefficient of the tracer.

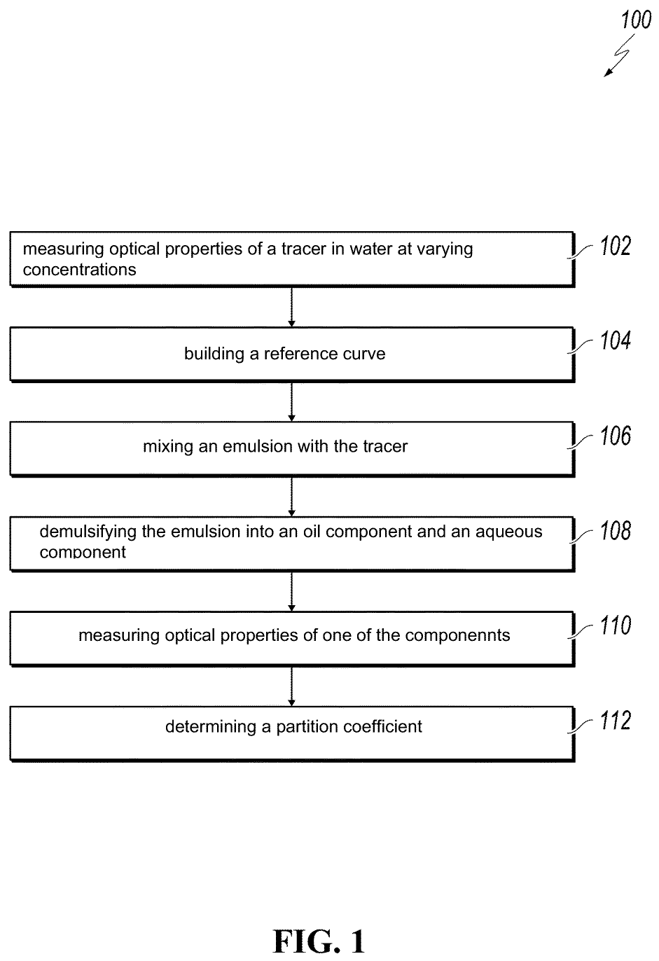

FIG. 1 is a flowchart of a method 100 that can be used with aspects of this disclosure. In particular, the methods described herein can be used to determine the partition coefficient of analytes on the order of minutes. At 102, optical properties of a tracer in water are measured at varying concentrations. In some implementations, optical properties can include visibility at various wavelengths, such as ultraviolet visibility or short, medium, and long-wave infrared. In some implementations, optical properties include fluorescence intensity, or amplitude, with or without stimulation. In instances where stimulation is used, stimulation can include stimulation by a wavelength of light, for example, stimulation light at 214 nanometer wavelength or 254 nanometer wavelength. Tracers that can be used with aspects of this disclosure include dipicolinic acid; chelidamic acid, 4-chloropyridine-2, 6-dicarboxylic acid, 1, 5-naphthalenedisulfonate, 2-fluorobenzoic acid, or, 4-Chlorobenzyl alcohol. Other tracers beyond those explicitly listed or described within this disclosure can be used without departing from this disclosure.

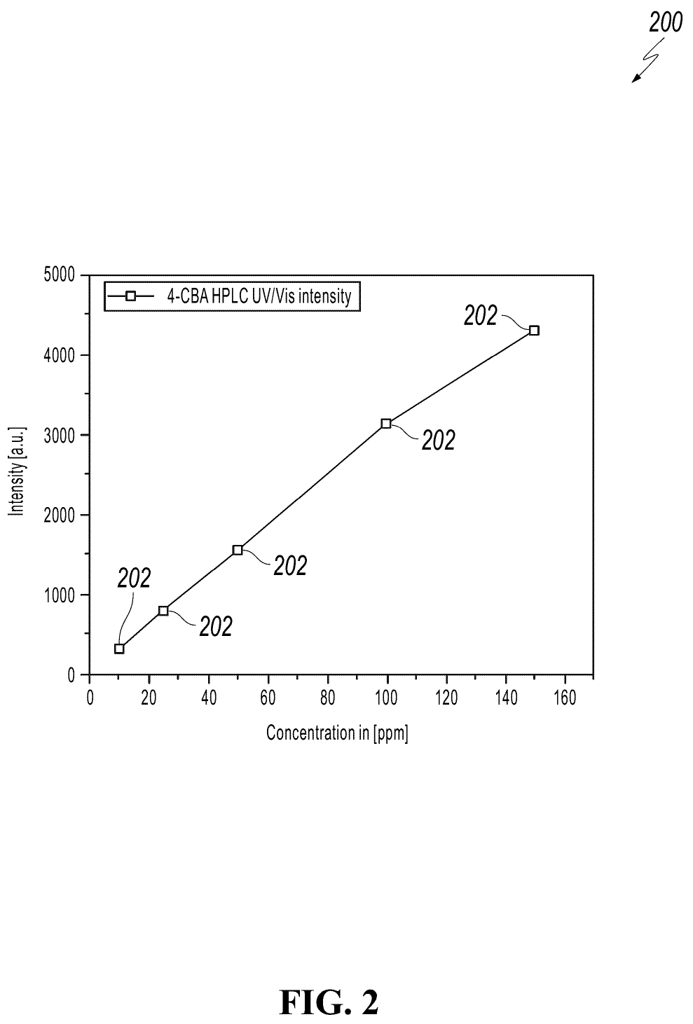

At 104, based on the measured optical properties at varying concentrations, a reference curve is built. FIG. 2 illustrates an example of such a reference curve 200. To develop this curve, a sample with a first amount of tracer is measured and recorded. Subsequent samples with varying concentrations are similarly measured to develop the reference points 202 seen on the curve 200. In some implementations, the tracer is an aqueous tracer mixed with water at various concentrations for each of the samples. This curve 200 is later used as a reference point for the remainder of the method steps. Method steps 102 and 104 can be performed in advance of the remaining steps to develop an initial reference curve 200. Multiple reference curves 200 for multiple tracers can be developed and stored for reference at a later date.

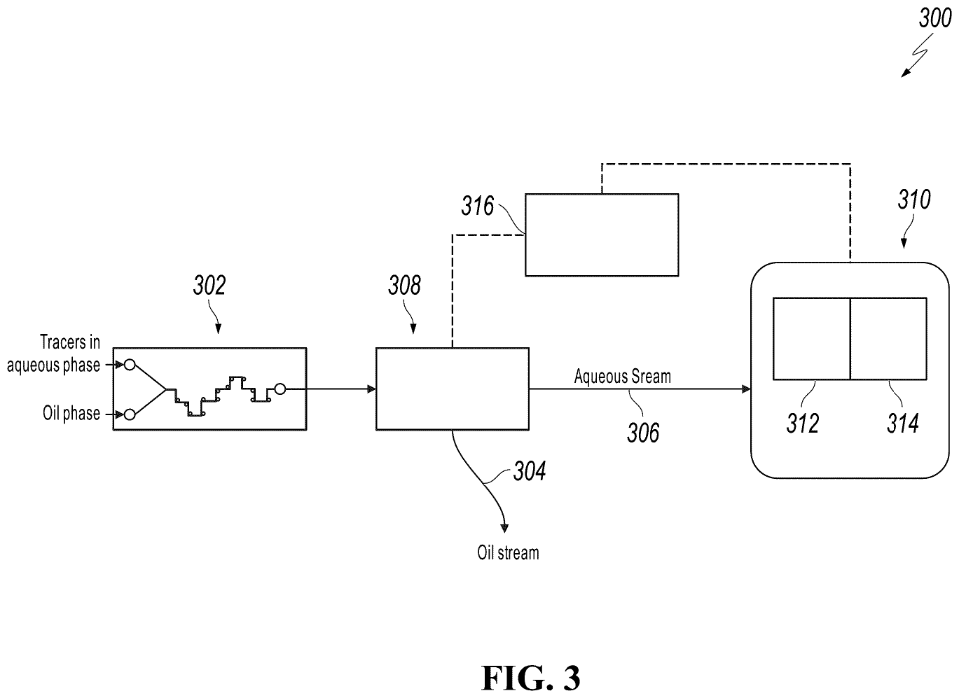

Referring back to FIG. 1, at 106, an emulsion and a tracer are mixed with one another. In some implementations, the tracer is an aqueous tracer. The remainder of the method 100 will be described in the context of FIG. 3, which is a schematic diagram of an example analysis system 300 that can be used with aspects of this disclosure.

In some implementations, a microfluidic chip 302 (see FIG. 3) can be used. While this disclosure primarily references and describes using a microfluidic chip for mixing, several different mixing techniques can be used without departing from this disclosure. For example, sonication or dynamic shakers can be used without departing from this disclosure.

In an example procedure, 3 ml of tracer dissolved water solution (100 ppm) is injected into a teardrop microfluidic mixing chip using a syringe pump with a flow rate 0.5 ml/min. Using a separate syringe pump, a 1 ml pulse of crude oil is concurrently injected with the aqueous solution. The various fluid components are thoroughly mixed in a microfluidic mixing chip. This example procedure makes apparent the small quantities of fluids needed to determine a partition coefficient.

After the emulsion and tracer are mixed with one another, at 108, the emulsion is demulsified into an oil component 304 and an aqueous component 306. In some implementations, demulsion is done mechanically. That is, additional chemical demulsifiers are not added. Rather, for example, a membrane separator 308 is used. In some implementations, other demulsification techniques can be used. For example, a hydrocyclone or centrifugal separator can be used to demulsify the mixture without departing from this disclosure.

At 110, optical properties of one of the components are measured. In the illustrated implementation, the water component is measured. Such a scenario can occur, for example, when an aqueous tracer is used. In some implementations, the oil component is measured. In such an implementation, the oil component 304 is directed into a measuring apparatus, or optical detector 310, in lieu of the aqueous component 306.

In some implementations, the optical properties are measured by an optical detection system 310. In some implementations, measuring optical properties includes exposing the component sample or stream to a specified wavelength of light. For example, the sample or stream can be exposed to light source 312 emitting light at 214 nanometer wavelength or 254 nanometer wavelength. In such implementations, a light source 312 emitting other specified wavelengths of light can be used. Implementations using such a light source can be used, for example, in instances where measuring the optical properties includes measuring a visibility or fluorescent intensity of the sample responsive to exposing the sample to the specified wavelength of light. To measure such properties, the optical detection system includes a light sensor 314. The light sensor can be tuned to a single wavelength of light or a range or wavelengths. Depending upon the configuration, the optical sensor can be adjacent to the light source 312, perpendicular to the light source, or directly across from the light source. Other relative orientations of the light source 312 and light sensor can be used without departing from this disclosure. Regardless of orientation, the light source 312 and light sensor 314 are both arranged to shine towards a sample or stream being measured.

At 112, a partition coefficient of the tracer is determined based on the measured optical properties of a demulsified component and the reference curve 200. For example, the partition coefficient can be determined by comparing the fluorescence intensity of the sample or stream to the curve using the following equations:

$${K}_{OW} = \frac{{L}_{W}}{{L}_{O}} [ \frac{{I}_{W , initial} - {I}_{dark}}{{I}_{W , final} - {I}_{dark}} - 1 ] \qquad ( 1 )$$

$${K}_{OW} = \frac{{C}_{O}}{{C}_{W}} \qquad ( 2 )$$

where, K_{ow} is the partition coefficient, Lₒ is a length of the octanol droplet in a microchannel of the microfluidic ship, L_{w} is the length of a water droplet in the microchannel, I_{w} is fluorescence intensity of the analytes in water, I_{dark} is the fluorescence intensity of the channel without analytes, Cₒ is the concentration of analytes in oil and C_{w} is the concentration of analytes in water. In some implementations, other optical properties and other equations can be used without departing from this disclosure. The optical detector 310, the membrane separator 308, or both, in some implementations, are coupled to a controller 316.

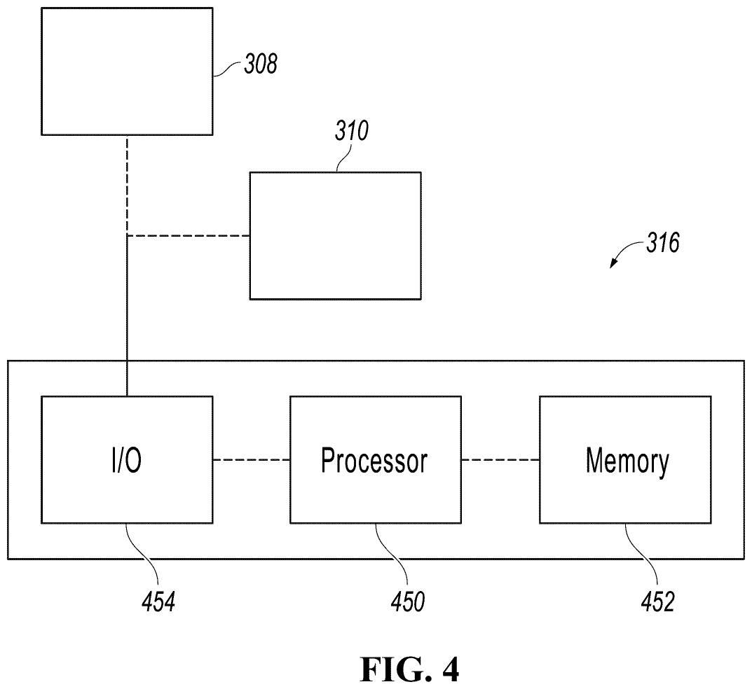

FIG. 4 is a schematic diagram of an example controller 316 that can be used with aspects of this disclosure. The controller 316 can, among other things, monitor parameters of the system 300 and send signals to actuate and/or adjust various operating parameters of the system 300. As shown in FIG. 4, the controller 316, in certain instances, includes a processor 450 (e.g., implemented as one processor or multiple processors) and a memory 452 (e.g., implemented as one memory or multiple memories) containing instructions that cause the processors 450 to perform operations described herein. The processors 450 are coupled to an input/output (I/O) interface 454 for sending and receiving communications with components in the system, including, for example, the light sensor 314. In certain instances, the controller 316 can additionally communicate status with and send actuation and/or control signals to one or more of the various system components (including an actuable systems, such as the light source 312 or the membrane separator 308) of the system 300, as well as other sensors (e.g., pressure sensors, temperature sensors, and other types of sensors) provided in the system 300. In certain instances, the controller 316 can communicate status and send control signals to one or more of the components within the system 300, such as the actuator light source 312. The communications can be hard-wired, wireless or a combination of wired and wireless. In some implementations, controllers similar to the controller 316 can be located elsewhere, such as in a data van, elsewhere on a site or even remote from the site. In some implementations, the controller 316 can be a distributed controller with different portions located about a site or off site. For example, in certain instances, the controller 316 can be located at the optical detector 310, or it can be located in a separate control room or data van. Additional controllers can be used throughout the site as stand-alone controllers or networked controllers without departing from this disclosure.

The controller 316 can operate in monitoring, commanding, and using the system 300 for measuring and determining partition coefficients of tracers. To make such determinations, the controller 316 is used in conjunction with the optical, or light sensor 314. Input and output signals, including the data from the sensor, controlled and monitored by the controller 316, can be logged continuously by the controller 316 within the controller memory 452 or at another location.

The controller 316 can have varying levels of autonomy for controlling the system 300. For example, the controller 316 can begin the method 100, and an operator adjusts the membrane separator 308 and optical detector 310. Alternatively, the controller 316 can begin the method 100, receive an additional input from an operator, and begin adjusting the membrane separator 308 and optical detector 310 with no other input from an operator. Alternatively, the controller 316 can begin the method 100 and adjust membrane separator 308 and optical detector 310 with no input from an operator.

Regardless of the autonomy of the controller operation, the controller can perform any of the following functions. The controller is configured to send a signal to the separator 308. The signal is a command to begin the separation process, demulsifying the emulsion. In some implementations, the controller 316 is configured to activate a light source emitting a light at 214 nanometer wavelength or 254 nanometer wavelength. A measurement signal is received by the controller 316 from an optical detector 310. The measurement signal is indicative of a concentration of a tracer within the measured sample or stream. The controller 316 then compares the measurement signal to a known dataset of tracer concentrations. The controller can then determine a partition coefficient based on the comparison of the measurement signal with the dataset.

While this disclosure contains many specific implementation details, these should not be construed as limitations on the scope of what may be claimed, but rather as descriptions of features specific to particular implementations. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the implementations described above should not be understood as requiring such separation in all implementations, and it should be understood that the described components and systems can generally be integrated together in a single product or packaged into multiple products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

1. A system comprising:
a microfluidic mixing chip having a first inlet and a second inlet;
a membrane separator connected and downstream to the microfluidic mixing chip;
an optical measurement system external to the microfluidic mixing chip; and
a controller configured to:
measure properties of tracer solutions comprising varying concentrations of a tracer;
provide a reference curve based on the measured optical properties of the tracer solutions; and
form a mixed emulsion by mixing a pulse of crude oil and an aqueous solution comprising a known concentration of the tracer, wherein the mixing comprises:
injecting the pulse of crude oil into the first inlet using a first syringe pump, and
injecting the aqueous solution into the second inlet using a second syringe pump; and
the controller further configured to:
demulsify the formed mixed emulsion into an oil component and an aqueous component by flowing the formed mixed emulsion through the membrane separator;
send the oil component or the aqueous component to the optical measurement system; and
measuring a component optical property of the oil component or of the aqueous component using the optical measurement system, the component optical property comprising a ultraviolet visibility or a fluorescence intensity, wherein the component optical property is indicative of a concentration of the tracer in the oil component or of a concentration of the tracer in the aqueous component; and
determine a partition coefficient of the tracer based on the measured component optical property and on the reference curve.

2. The system of claim 1, wherein the controller is further configured to:
activate a light source emitting a light at 214 nanometer wavelength or 254 nanometer wavelength.

3. The system of claim 1, wherein the optical measurement system comprises a light sensor.

4. The system of claim 1, wherein the optical measurement system comprises a controllable light source emitting light at 254 nanometer wavelength.

5. The system of claim 1, wherein the tracer comprises: dipicolinic acid; chelidamic acid; 4-chloropyridine-2; 6-dicarboxylic acid; 1, 5-naphthalenedisulfonate; 2-fluorobenzoic acid; or 4-Chlorobenzyl alcohol.

6. A system of determining a partition coefficient of a tracer, the system comprising:
a microfluidic mixing chip having a first inlet and a second inlet;
a membrane separator connected and downstream to the microfluidic mixing chip;
an optical measurement system external to the microfluidic mixing chip; and
a controller configured to:
measure optical properties of aqueous tracer solutions comprising varying concentrations of a tracer;
develop a reference curve based on the measured optical properties of the aqueous tracer solutions;
form a an emulsion by mixing a pulse of crude oil and an aqueous solution comprising a known concentration of the tracer, the mixing comprising injecting 1 mL of the pulse of crude oil into the first inlet, and injecting 3 mL of the aqueous solution into the second inlet;
form a demulsified component by demulsifying the formed emulsion using the membrane separator;
send the demulsified component to the optical measurement system;
measure an optical property of the demulsified component using the optical measurement system, the optical property comprising a ultraviolet visibility or a fluorescence intensity and indicative of a concentration of the tracer in the demulsified component; and
determine a partition coefficient of the tracer based on the measured optical property of the demulsified component and the developed reference curve.

7. The system of claim 6, wherein the measuring the optical property of the demulsified component comprises exposing the demulsified component to a light at having a 214 nanometer wavelength or a 254 nanometer wavelength.

8. The system of claim 7, wherein the measuring the optical property of the demulsified component comprises measuring a visibility or a fluorescent intensity of the demulsified component responsive to the exposing.

9. The system of claim 6, wherein the tracer comprises: dipicolinic acid; chelidamic acid; 4-chloropyridine-2; 6-dicarboxylic acid; 1, 5-naphthalenedisulfonate; 2-fluorobenzoic acid; or 4-Chlorobenzyl alcohol.

10. The system of claim 6, wherein the demulsified component is an aqueous component.

11. The system of claim 6, wherein the demulsified component is an oil component.

12. The system of claim 6, wherein the optical property comprises a fluorescence intensity.

13. The system of claim 6, wherein demulsifying the formed mixed emulsion is performed mechanically.
